(19) United States
(12) Patent Application Publication
(10) Pub. No.: US 2011/0237605 A1
(43) Pub. Date: Sep. 29, 2011

(54) Molecular Crystal of (4-(1,8-Naphthyridin-2-YL)Piperidin-1-YL)Pyrimidine Derivative

(76) Inventors: Eric Phillips, Ontario, NY (US); Mohannad Shawer, Webster, NY (US)

(21) Appl. No.: 13/018,896

(22) Filed: Feb. 1, 2011

Related U.S. Application Data
(60) Provisional application No. 61/317,942, filed on Mar. 26, 2010.

(51) Int. Cl. A61K 31/506; C07D 471/04; A61P 27/02; A61P 35/00

(52) U.S. Cl. 514/256; 544/322

(57) ABSTRACT

A molecular crystal form of (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid is characterized by an X-ray powder diffraction (“XRPD”) spectrum that comprises peaks at 2θ angles of 7.44, 14.80, 16.64, and 23.04±0.2°. The molecular crystal can be formulated into pharmaceutical composition for treating or controlling diseases resulting from pathological angiogenesis.

### CROSS REFERENCE

This application claims the benefit of Provisional Patent Application No. 61/317,942 filed Mar. 26, 2010 which is incorporated by reference herein.

### BACKGROUND OF THE INVENTION

The present invention relates to a molecular crystal of a (4-(1,8-naphthyridin-2-yl)piperidin-1-yl)pyrimidine derivative. The present invention also relates to methods of making and using such molecular crystal.

(4-(1,8-naphthyridin-2-yl)-piperidin-1-yl)pyrimidine derivatives have been disclosed in U.S. Pat. No. 7,582,640; US Patent Application Publication 2008/0058348; and PCT Patent Application Publication WO 2005/123734 as candidates for use as antagonists of vitronectin receptor. However, these documents do not disclose any particular molecular crystal form of any of the exemplified compounds.

Active pharmaceutical agents (“APIs”) are often organic molecules, which can exist in different organic crystal forms depending on their processes of manufacture. Such different molecular crystal forms can have practical influence on pharmaceutical compositions comprising these APIs, such as their processability, physical and chemical properties, stability, etc.

Therefore, it is desirable to provide a molecular crystal form of the API that has advantageous properties. In particular, it is very desirable to provide a molecular crystal form of a (4-(1,8-naphthyridin-2-yl)-piperidin-1-yl)pyrimidine derivative that has advantageous properties for the manufacture of novel pharmaceutical compositions for the inhibition of activity of vitronectin receptor.

### SUMMARY

In general, the present invention provides a specific molecular form of a (4-(1,8-naphthyridin-2-yl)-piperidin-1-yl)pyrimidine derivative.

In one aspect, the present invention provides a specific molecular form of (4-(1,8-naphthyridin-2-yl)-piperidin-1-yl)pyrimidine derivative having Formula I.

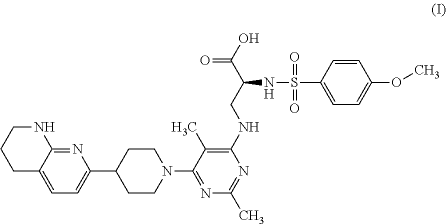

A chemical name of the (4-(1,8-naphthyridin-2-yl)-piperidin-1-yl)pyrimidine derivative having Formula I is (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid. The compound having Formula I is also known under an alternative name of 3-(2,5-dimethyl-6-(4-(5,6,7,8-tetrahydro-1,8-naphthyridin-2-yl)piperidin-1-yl)pyrimidin-4-ylamino)-N-(4-methoxyphenylsulfonyl)(L)-alanine.

In another aspect, the present invention provides a stable molecular form of the compound having Formula I. As used herein, the term “stable molecular fond” of a compound means that the compound is incapable, or substantially incapable, of changing in crystalline structure, as exhibited by a plurality of peaks in an X-ray powder diffraction (“XRPD”) spectrum (or sometimes alternatively called “pattern”), upon storage at normal room conditions of temperature, pressure, and humidity after at least 1 month, as exhibited by a relative change of less than 5 percent (or less than about 5 percent) in the peak height of the highest peak in its XRPD spectrum. Alternatively, molecular crystal is stable when the positions of the plurality of major peaks, as exhibited by the 2θangles, of the XRPD spectrum do not change within the experimental uncertainty of the

In still another aspect, such normal room conditions of temperature, pressure, and humidity are 20-28° C., 95-105 kPa, and 20-80% relative humidity.

In yet another aspect, the present invention provides a molecular crystal form of (4-(1,8-naphthyridin-2-yl)-piperidin-1-yl)pyrimidine derivative having Formula I characterized by an X-ray powder diffraction (“XRPD”) spectrum that comprises peaks at 2θ angles of 7.44, 14.80, 16.64, and 23.04±0.2°.

In yet another aspect, the present invention provides a molecular crystal form of (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid characterized by an XRPD spectrum that comprises peaks at 2θ angles of 7.44, 14.80, 16.64, 23.04, and 23.36±0.2°.

In a further aspect, the present invention provides a process for preparing or producing said stable molecular crystal. The process comprising subjecting an aqueous suspension of (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid to an autoclaving condition at 121-125° C., a pressure of about 100-120 kPa (e.g., about 100 kPa) above atmospheric pressure, for 30 minutes to 10 hours, under a closed atmosphere generated by said aqueous suspension.

In an embodiment, said pressure is about 200-220 kPa.

In still another aspect, the present invention provides a pharmaceutical composition comprising said stable molecular crystal of said (4-(1,8-naphthyridin-2-yl)-piperidin-1-yl)pyrimidine derivative having Formula I.

Other features and advantages of the present invention will become apparent from the following detailed description and claims and the appended drawings.

### BRIEF DESCRIPTION OF THE DRAWINGS

FIG. 1 shows an XRPD spectrum of a molecular crystal of (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid.

FIG. 2 shows an XRPD spectrum of a molecular crystal of (5)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid of the present invention.

FIG. 3 shows the particle distributions of the molecular crystal, the XRPD spectrum of which is shown in FIG. 1, and of the molecular crystal of the present invention, the XRPD spectrum of which is shown in FIG. 2.

FIG. 4 shows XRPD spectra of the original compound having Formula I, the claimed molecular crystal, and the same subject to further milling and/or autoclaving. A comparison of the spectra indicates that the claimed molecular crystal form is stable.

### DETAILED DESCRIPTION OF THE INVENTION

As used herein, the term “control” also includes reduction, alleviation, and amelioration. The term “control,” when associated with a patient, who is at risk of developing a disease, also includes prevention.

In general, the present invention provides a specific molecular form of a (4-(1,8-naphthyridin-2-yl)-piperidin-1-yl)pyrimidine derivative.

In one aspect, the present invention provides a specific molecular form of (4-(1,8-naphthyridin-2-yl)-piperidin-1-yl)pyrimidine derivative having Formula I.

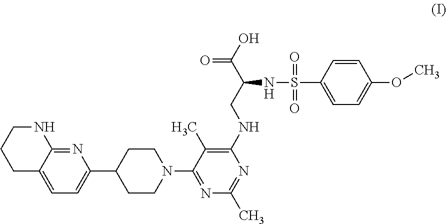

A chemical name of the (4-(1,8-naphthyridin-2-yl)-piperidin-1-yl)pyrimidine derivative having Formula I is (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid. The compound having Formula I is also known under an alternative name of 3-(2,5-dimethyl-6-(4-(5,6,7,8-tetrahydro-1,8-naphthyridin-2-yl)piperidin-1-yl)pyrimidin-4-ylamino)-N-(4-methoxyphenylsulfonyl)L-alanine.

In another aspect, the present invention provides a stable molecular form of the compound having Formula I.

In a further aspect, the present invention provides a process for preparing said stable molecular crystal. The process comprising subjecting an aqueous suspension of a starting (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid to an autoclaving condition at 121-125° C., about 100 kPa above atmospheric pressure, for 30 minutes to 10 hours (or alternatively, 30 minutes to 8 hours, 30 minutes to 6 hours, 30 minutes to 4 hours, 30 minutes to 2 hours, 30 minutes to 1 hour), under a closed atmosphere generated by said aqueous suspension.

In one aspect, said starting is (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid can comprise, for example, an amorphous material.

In another aspect, said starting is (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid can comprise, for example, a crystalline form that can further be converted to the crystalline form of the present invention upon being subject to an autoclaving condition. In one embodiment, such autoclaving condition is disclosed hereinabove.

In another aspect, the starting material, without restriction as to the physical or chemical stability of its form, can be prepared according a process disclosed in U.S. Pat. No. 7,582,640 and US Patent Application Publication 2010/0041675, which are incorporated herein by reference in their entireties.

Synthesis of (5)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid starting compound for the production of the claimed crystalline material.

First, 2,5-dimethyl-4,6-dihydroxy-pyrimidine is reacted with hydrochloric acid or SO₂Cl in pyridine to produce 2,5-dimethyl-4,6-dichloro-pyrimidine.

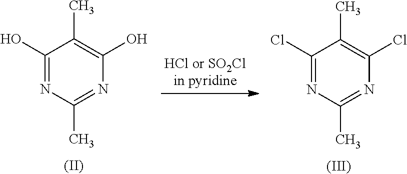

Then, 2,5-dimethyl-4,6-dichloro-pyrimidine is reacted with (L)-2,3-diaminopropionic acid to produce ((2,5-dimethyl-6-chloro)pyrimidin-4-ylamino)(L)-2-aminopropionic acid ester.

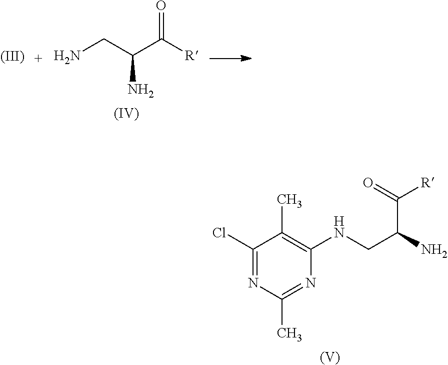

wherein —C(O)R′ is a hindered ester group, such as the t-butyloxycarbonyl group. In general, a hindered strong base is used as a component of the reaction medium, such as diisopropylethylamine, under reaction conditions known to a person skilled in the art for the implementation of nucleophilic substitution. Preferably, the operation takes place in the presence of dimethylformamide and under reflux, at a temperature up to the boiling point of the solvent.

Then the compound having Formula V is reacted with 4-(5,6,7,8-tetrahydro-1,8-napthyridin-2-yl)piperidine (having Formula VI) to produce the compound having Formula VII ((2,5-dimethyl-6-(4-(5,6,7,8-tetrahydro-1,8naphthyridin-2-yl)piperidin-1-yl)pyrimidin-4-ylamino)(L)-2-aminopropionic acid ester). 4-(5,6,7,8-tetrahydro-1,8-napthyridin-2-yl)piperidine can be prepared by a method disclosed in U.S. Pat. No. 6,743,800; EP1065207; or WO00/78317, which are incorporated herein by reference in their entireties.

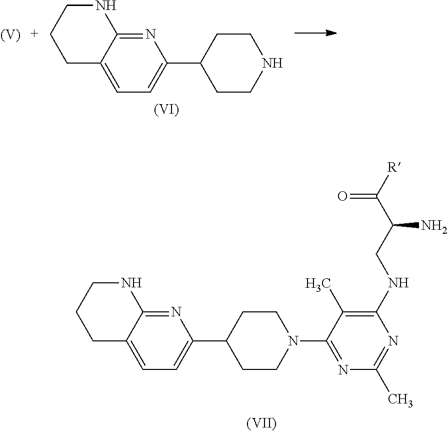

For example, a quantity of the compound VI is added to a 50/50 (% by volume) mixture of dichloromethane and methanol. A stoichiometric quantity of compound V in diisopropylethylamine is added to the solution of compound VI in dichloromethane/methanol mixture. The reaction is carried out under reflux for about 4-12 hours. The reaction mixture is evaporated to dryness under vacuum to recover a solid including compound VII. This solid is dissolved in an appropriate solvent, such as aqueous ethyl acetate, and compound VII is separated by, for example, chromatography using dichloromethane eluent.

Compound IX is prepared by reacting compound VII and compound VIII ((4-methoxy)phenylsulfonyl chloride) in a mixture of triethylamine (“TEA”, such as 2-15 parts by volume) and tetrahydrofuran (“THF”, such as 85-98 parts by volume) under vigorous stirring at room temperature (or within 10° C. above room temperature) for 1-10 hours according to the reaction disclosed below.

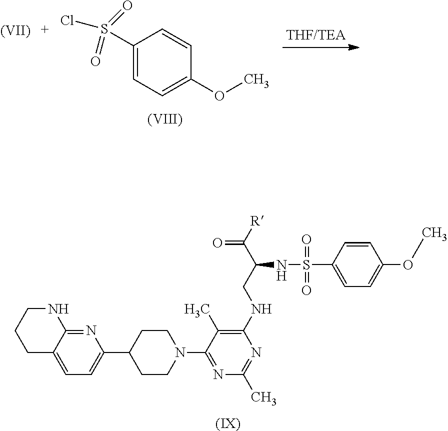

The reaction mixture is evaporated to dryness under vacuum (such as 2 kPa) The resulting solid is dissolved in an aqueous mixture of ethyl acetate, water, and saturated sodium bicarbonate. The organic phase is separated and dried over magnesium sulfate, and the solvent is evaporated under vacuum (such as 2 kPa). Compound IX is separated by chromatography.

Compound I is obtained from compound IX by hydrolysis. For example, a quantity of compound IX is stirred in a mixture of dichloromethane (for example, 8-9 parts by volume) and trifluoroacetic acid (for example, 1-2 parts by volume) at room temperature until all the starting material of compound IX visually disappears. Toluene is then added under stirring, and the mixture is evaporated to dryness under vacuum (e.g., 2 kPa). The resulting residue is dissolved in a minimum amount of dichloromethane with a small amount of methanol. The mixture is poured into diisopropyl ether. The precipitate is filtered and washed with water to yield compound I. The purity of compound I can be further enhanced by chromatography.

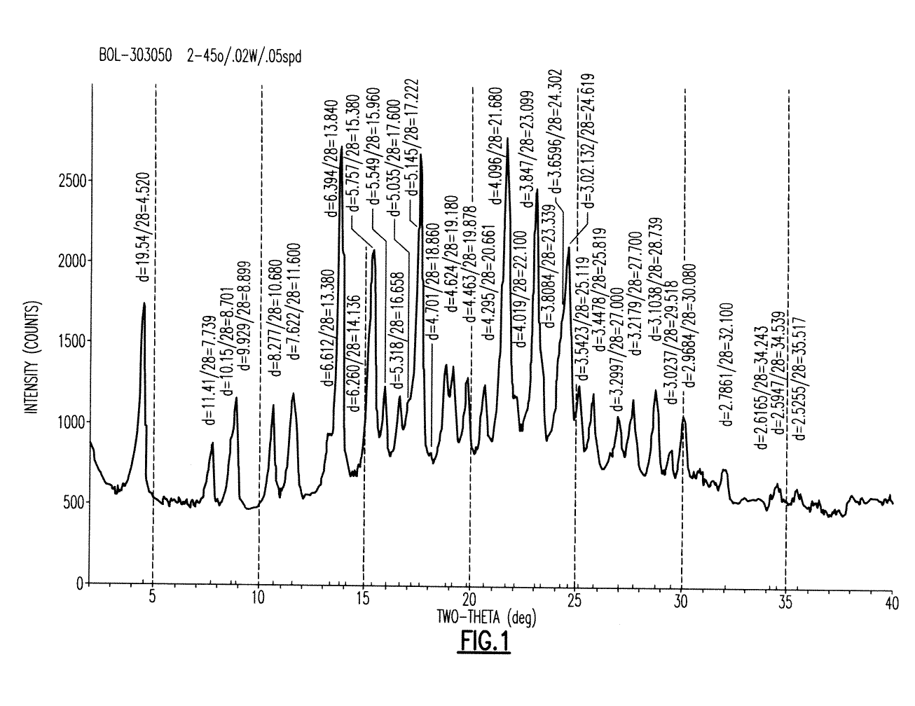

An XRPD spectrum of compound I, before any further novel processing as disclosed and claimed herein, is shown in FIG. 1. Identifiable peaks of the spectrum of FIG. 1 are shown in Table 1.

TABLE 1
XRD Peaks of Spectrum Shown in FIG. 1

| | |
|---|---|
| [0371.raw] BOL-303050-2-45o/.02SW/.05spd | Peak Search Report |

SCAN: 2.0/60.0/0.02/2.4 (sec). Cu(30 kV, 15 mA). I(max) = 2779. Feb. 16, 2009 01:49 p
PEAK: 29 pts/Quartic Filter, Threshold = 3.0, Cutoff = 0.1%, BG = 1/1.0, Peak-Top = Summit

| # | 2-Theta | d(A) | BG | Height | H% | Area | A% | FWHM |
|---|---|---|---|---|---|---|---|---|
| 1 | 4.520 | 19.5355 | 530 | 1202 | 57.7 | 23276 | 54.8 | 0.329 |
| 2 | 7 739 | 11.4145 | 488 | 379 | 18.2 | 6380 | 15.0 | 0.266 |
| 3 | 8.701 | 10.1546 | 481 | 433 | 20.8 | 12904 | 30.4 | 0.506 |
| 4 | 8.899 | 9.9286 | 478 | 668 | 32.1 | 13236 | 31.2 | 0.337 |
| 5 | 10 680 | 8.2768 | 530 | 581 | 27.9 | 9147 | 21.6 | 0.267 |
| 6 | 11.600 | 7.6224 | 544 | 639 | 30.7 | 13512 | 31.8 | 0.360 |
| 7 | 13.380 | 6.6120 | 615 | 324 | 15.6 | 9227 | 21.7 | 0.484 |
| 8 | 13 840 | 6.3935 | 641 | 2084 | 100.0 | 38911 | 91.7 | 0.317 |
| 9 | 14.136 | 6.2603 | 698 | 84 | 4.1 | 2638 | 6.2 | 0.531 |
| 10 | 15.380 | 5.7565 | 739 | 1335 | 64.1 | 29710 | 70.0 | 0.378 |
| 11 | 15 960 | 5.5487 | 814 | 418 | 20.0 | 6057 | 14.3 | 0.247 |
| 12 | 16.658 | 5.3175 | 921 | 252 | 12.1 | 2744 | 6.5 | 0.185 |
| 13 | 17.222 | 5.1449 | 784 | 492 | 23.6 | 30844 | 72.7 | 1.066 |
| 14 | 17 600 | 5.0352 | 857 | 1818 | 87.2 | 32718 | 77.1 | 0.306 |
| 15 | 18.860 | 4.7015 | 825 | 549 | 26.4 | 15245 | 35.9 | 0.472 |
| 16 | 19.180 | 4.6237 | 788 | 572 | 27.4 | 21603 | 50.9 | 0.642 |
| 17 | 19.878 | 4.4628 | 851 | 443 | 21.3 | 6139 | 14.5 | 0.236 |
| 18 | 20.661 | 4.2955 | 867 | 382 | 18.3 | 5573 | 13.1 | 0.248 |
| 19 | 21.680 | 4.0968 | 938 | 1841 | 88.4 | 42446 | 100.0 | 0.392 |
| 20 | 22.100 | 4.0189 | 965 | 220 | 10.5 | 11538 | 27.2 | 0.893 |
| 21 | 23.099 | 3.8473 | 946 | 1520 | 73.0 | 29350 | 69.1 | 0.328 |
| 22 | 23.339 | 3.8084 | 933 | 492 | 23.6 | 19595 | 46.2 | 0.677 |
| 23 | 24.302 | 3.6596 | 923 | 737 | 35.4 | 32455 | 76.5 | 0.749 |
| 24 | 24.619 | 3.6132 | 877 | 1232 | 59.1 | 39289 | 92.6 | 0.542 |
| 25 | 25.119 | 3.5423 | 1003 | 248 | 11.9 | 1935 | 4.6 | 0.133 |
| 26 | 25.819 | 3.4478 | 790 | 407 | 19.5 | 6205 | 14.6 | 0.259 |
| 27 | 27.000 | 3.2997 | 732 | 327 | 15.7 | 8501 | 20.0 | 0.442 |
| 28 | 27.700 | 3.2179 | 769 | 397 | 19.0 | 6035 | 14.2 | 0.259 |
| 29 | 28.739 | 3.1038 | 691 | 533 | 25.6 | 11316 | 26.7 | 0.361 |
| 30 | 29.518 | 3.0237 | 697 | 149 | 7.2 | 2182 | 5.1 | 0.249 |
| 31 | 30.080 | 2.9684 | 680 | 379 | 18.2 | 7375 | 17.4 | 0.331 |
| 32 | 32.100 | 2.7861 | 569 | 168 | 8.1 | 2692 | 6.3 | 0.272 |

NOTE:
Intensity = Counts,
2T(0) = 0.0 (deg),
Wavelength to Compute d-Spacing = 1.54059A (Cu/K-alpha1)

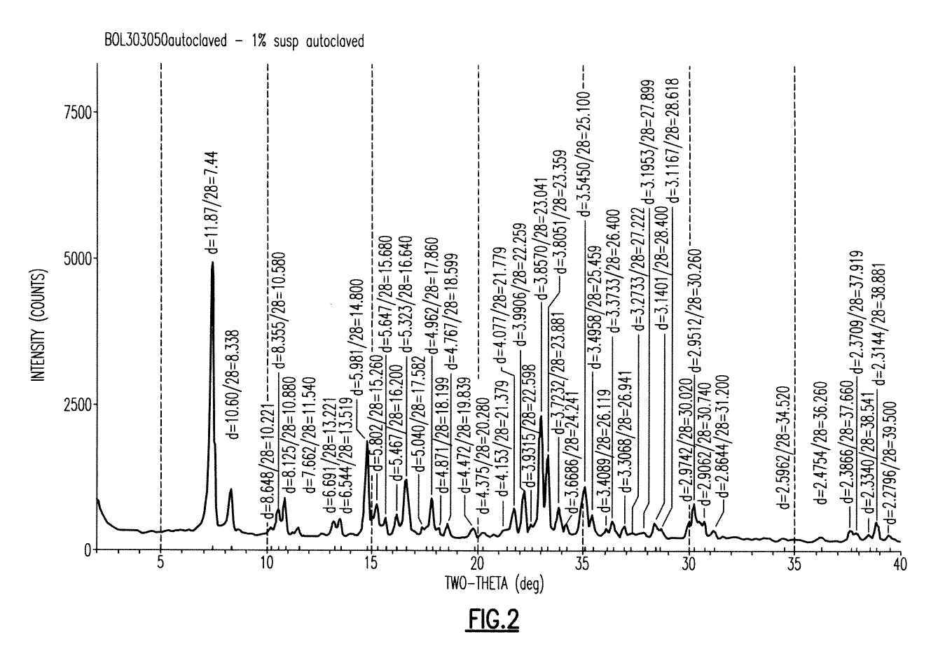

A suspension of a quantity of compound I was further autoclaved under autoclaving condition at 121-125° C., about 100 kPa above atmospheric pressure, for 30 minutes to 10 hours, under a closed atmosphere generated by said aqueous suspension to yield the claimed stable molecular crystal, which has a unique XRPD spectrum as shown in FIG. 2. Identifiable peaks of the spectrum of FIG. 2 are shown in Table 2.

TABLE 2
XRD Peaks of Spectrum Shown in FIG. 2

| | |
|---|---|
| [0392.raw] BOL303050autoclaved-1% susp autoclaved | Peak Search Rep |

SCAN: 2.0/40.0/0.02/2.4 (sec), Cu(30 kV, 15 mA), I(max) = 6036. Mar. 6, 2009 01:05 p
PEAK: 29 pts/Quartic Filter, Threshold = 3.0, Cutoff = 0.1%, BG = 1/1.0, Peak-Top = Summit

| # | 2-Theta | d(A) | BG | Height | H% | Area | A% | FWHM |
|---|---|---|---|---|---|---|---|---|
| 1 | 7.441 | 11.8714 | 472 | 5564 | 100.0 | 71195 | 100.0 | 0.218 |
| 2 | 8.338 | 10.5954 | 452 | 814 | 14.6 | 8666 | 12.2 | 0.181 |
| 3 | 10.221 | 8.6476 | 329 | 154 | 2.8 | 4640 | 6.5 | 0.513 |
| 4 | 10.580 | 6.3553 | 331 | 614 | 9.2 | 11236 | 15.8 | 0.371 |
| 5 | 10.880 | 8.1248 | 320 | 763 | 13.7 | 12971 | 18.2 | 0.289 |
| 6 | 11.540 | 7.6622 | 311 | 167 | 3.0 | 1874 | 2.6 | 0.191 |
| 7 | 13.221 | 6.6915 | 303 | 300 | 5.4 | 6061 | 8.5 | 0.343 |
| 8 | 13.519 | 6.5443 | 305 | 349 | 6.3 | 7743 | 10.9 | 0.377 |
| 9 | 14.800 | 5.9808 | 331 | 1962 | 35.3 | 25223 | 35.4 | 0.219 |
| 10 | 15.260 | 5.8016 | 332 | 632 | 11.4 | 15682 | 22.0 | 0.422 |
| 11 | 15.680 | 5.6470 | 410 | 256 | 4.6 | 2151 | 3.0 | 0.143 |
| 12 | 16.200 | 5.4669 | 343 | 395 | 7.1 | 6857 | 9.6 | 0.295 |
| 13 | 16.640 | 5.3234 | 345 | 1126 | 20.2 | 18839 | 26.5 | 0.284 |
| 14 | 17 582 | 5.0403 | 341 | 149 | 2.7 | 6002 | 8 4 | 0 686 |
| 15 | 17.860 | 4.9625 | 327 | 755 | 13.6 | 11811 | 16.6 | 0.266 |
| 16 | 18.199 | 4.8707 | 311 | 165 | 3.0 | 5208 | 7.3 | 0.536 |
| 17 | 18 599 | 4.7669 | 308 | 252 | 4 5 | 2330 | 3 3 | 0 157 |
| 18 | 19.839 | 4.4716 | 278 | 191 | 3.4 | 2665 | 3.7 | 0.237 |
| 19 | 20.280 | 4.3753 | 287 | 93 | 1.7 | 1567 | 2.2 | 0.285 |
| 20 | 21.379 | 4.1529 | 360 | 87 | 1.6 | 2125 | 3.0 | 0.414 |
| 21 | 21.779 | 4.0774 | 388 | 491 | 8.8 | 7207 | 10.1 | 0.249 |
| 22 | 22.259 | 3.9906 | 417 | 834 | 15.0 | 9002 | 12.6 | 0.183 |
| 23 | 22.598 | 3.9315 | 427 | 137 | 2.5 | 2507 | 3.5 | 0.312 |
| 24 | 23.041 | 3.8570 | 437 | 2392 | 43.0 | 28608 | 40.2 | 0.203 |
| 25 | 23.359 | 3.8051 | 404 | 1604 | 28.8 | 19990 | 28.1 | 0.212 |
| 26 | 23.881 | 3.7232 | 427 | 474 | 8.5 | 6062 | 8.5 | 0.217 |
| 27 | 24.241 | 3.6686 | 419 | 140 | 2.5 | 778 | 1.1 | 0.095 |
| 28 | 25.100 | 3.5450 | 338 | 997 | 17.9 | 17209 | 24.2 | 0.293 |
| 29 | 25.459 | 3.4958 | 329 | 406 | 7.3 | 6700 | 9.4 | 0.280 |
| 30 | 26.119 | 3.4089 | 318 | 151 | 2.7 | 2698 | 3.8 | 0.304 |
| 31 | 26.400 | 3.3733 | 316 | 300 | 5.4 | 5347 | 7.5 | 0.303 |
| 32 | 26.941 | 3.3068 | 329 | 190 | 3.4 | 1912 | 2.7 | 0.171 |

NOTE:
Intensity = Counts,
2T(0) = 0.0 (deg),
Wavelength to Compute d-Spacing = 1.54059A (Cu/K-alpha1)

It was demonstrated that the claimed molecular crystal comprises a stable materials as exhibited by the XRPD spectra of the foregoing autoclaved material which was subject to further milling and/or autoclaving. The following experiment evidences such stability of the claimed molecular crystal.

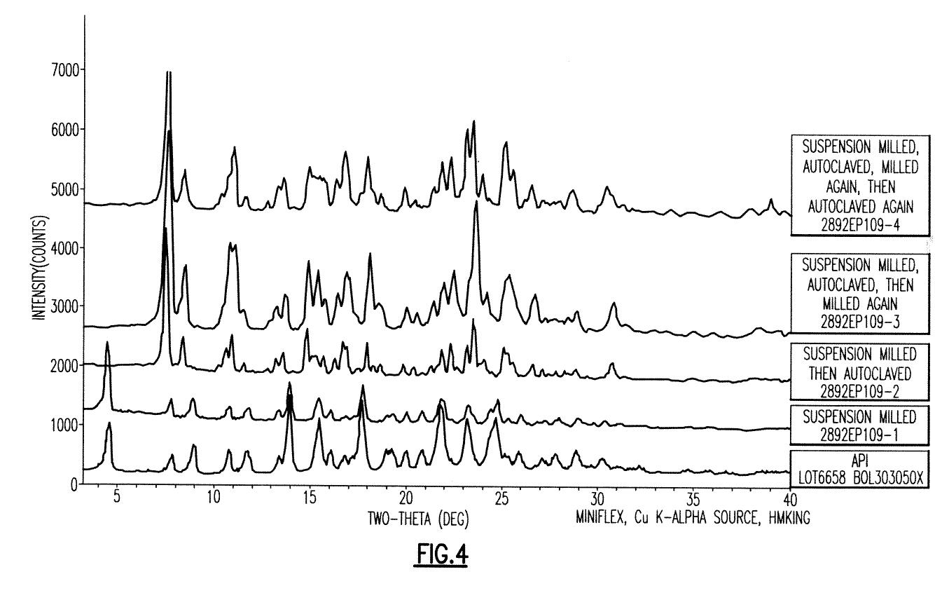

A 30 mg/ml suspension of the compound having Formula I (referred to as “BOL-303050”) was made in PBS-0.15% Polysorbate 80 and put in a vial. The contents of the vial were milled by 1 mm zirconia milling beads for 5 times, 2 minutes each at 2400 rpm in a DAC 150 FV Speedmixer and shaken to suspend the drug. An aliquot was withdrawn for XRPD (see FIG. 4, suspension milled). The withdrawn aliquot was centrifuged and dried before XRPD scan.

The remainder of the vial contents was autoclaved at 121° c. for 30 minutes. After autoclaving another sample was removed for XRPD (see FIG. 4, suspension milled and autoclaved). Sample was centrifuged and dried before XRPD scan.

To the remaining sample, 1 mm zirconia milling beads were added and the sample was milled 5 times, for 2 minutes each at 2400 rpm in a DAC 150 FV Speedmixer. After bead milling, a sample was removed for XRPD (see FIG. 4, suspension milled, autoclaved then milled again). Sample was centrifuged and dried before XRPD scan.

The remainder was autoclaved at 121° C. for 30 minutes (see FIG. 4, suspension milled, autoclaved, milled again, then autoclaved). Sample was centrifuged and dried before XRPD scan.

Samples were compared to the XRPD spectrum of the original API sample.

### Results:

As shown by XRPD (see FIG. 4), the polymorphic changes occurred only during the initial autoclave cycle. Milling the drug particle for the duration stated above, did not affect the drug polymorph. Subsequent autoclaving did not affect the claimed novel drug polymorph indicating that this novel polymorph can have higher physical stability and more suitable for pharmaceutical development of suspensions.

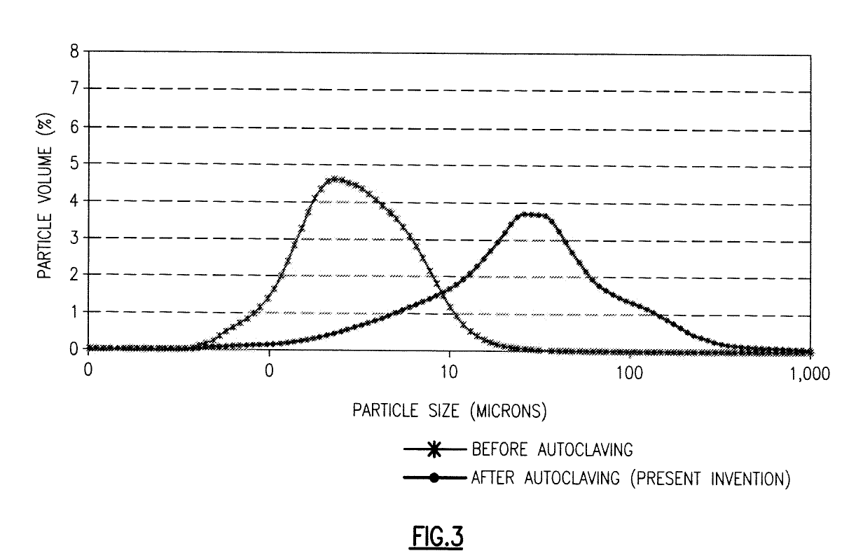

The particle distributions of the solid forms of compound I before and after autoclaving are shown in FIG. 3.

A non-limiting example of a process of preparing 4-(5,6,7,8-tetrahydro-1,8-napthyridin-2-yl)piperidine (compound VI) is disclosed immediately below.

### 4-(1,8-naphthyridin-2-yl)piperidine-1-carboxylic acid t-butyl ester

1-t-butyloxycarbonyl-4-acetyl-piperidine and 2-amino-3-formyl-pyridine is refluxed with L-proline in n-butanol for 4-72 hours. After removing the solvent in vacuo, the residue is chromatographed on silica gel with ethyl acetate/n-heptane (1:1) to give 4-(1,8-naphthyridin-2-yl)piperidine-1-carboxylic acid t-butyl ester.

### 4-(5,6,7,8-tetrahydro-1,8-naphthyridin-2-yl)piperidine-1-carboxylic acid t-butyl ester

A quantity of 4-(1,8-naphthyridin-2-yl)piperidine-1-carboxylic acid t-butyl ester is dissolved in ethyl acetate, and small amount (for example, about one part by weight per 5 parts by weight of the ester) of 10% palladium on charcoal catalyst is added under an inert gas atmosphere. Hydrogenation was performed with this mixture under stirring at ambient temperature until thin layer chromatography no longer shows the starting material. The catalyst is removed carefully and washed twice with ethyl acetate. The combined solutions are filtered again and the solvents removed in vacuo to yield 4-(5,6,7,8-tetrahydro-1,8-naphthyridin-2-yl)piperidine-1-carboxylic acid t-butyl ester.

### 4-(5,6,7,8-tetrahydro-1,8-naphthyridin-2-yl)piperidine

A quantity of 4-(5,6,7,8-tetrahydro-1,8-naphthyridin-2-yl)piperidine-1-carboxylic acid t-butyl ester is dissolved in an amount of methylene chloride (about 20-25 times the weight of said ester), and trifluoroacetic acid (about 4-6 times the weight of said ester) is added under stirring. Stirring is continued for 2-4 hours at room temperature. After removal of the solvents in vacuo, the oily residue is triturated with diethyl ether to yield 4-(5,6,7,8-tetrahydro-1,8-naphthyridin-2-yl)piperidine solid.

In still another aspect, the present invention provides a pharmaceutical composition comprising a stable molecular crystal of molecular crystal of said (4-(1,8-naphthyridin-2-yl)-piperidin-1-yl)pyrimidine derivative having Formula I.

In yet another aspect, the present invention provides a pharmaceutical composition comprising a stable molecular crystal form of (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid characterized by an X-ray powder diffraction (“XRPD”) spectrum that comprises peaks at 20 angles of 7.44, 14.80, 16.64, and 23.04±0.2°.

In a further aspect, the present invention provides a pharmaceutical composition comprising a stable molecular crystal form of (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid characterized by an X-ray powder diffraction (“XRPD”) spectrum that comprises peaks at 2θ angles of 7.44, 14.80, 16.64, 23.04, and 23.36±0.2°.

A pharmaceutical composition of the present invention can be used to treat a disease that is promoted by activity of vitronectin receptor, by administering such a composition to a subject who is at risk to develop, or suffers from, such disease.

In one aspect, such disease is selected from diseases that result from aberrant or pathological angiogenesis.

In another aspect, such disease is selected from the group consisting of age-related macular degeneration (“AMD”, including the wet and dry types), macular edema (including diabetic macular edema), diabetic retinopathy, and combinations thereof.

In still another aspect, the present invention provides a pharmaceutical composition comprising a stable molecular crystal form of (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid characterized by an X-ray powder diffraction (“XRPD”) spectrum that comprises peaks at 2θ angles of 7.44, 14.80, 16.64, 23.04, and 23.36±0.2° for the treatment or control of AMD.

In yet another aspect, the present invention provides a pharmaceutical composition comprising a stable molecular crystal form of (S)-3-(2,5-dimethyl-6-(4-(5,6,7,8-tetrahydro-1,8-naphthyridin-2-yl)piperidin-1-yl)pyrimidin-4-ylamino)-2-(4-methoxyphenylsulfonamido)propanoic acid characterized by an X-ray powder diffraction (“XRPD”) spectrum that comprises peaks at 2θ angles of 7.44, 14.80, 16.64, 23.04, and 23.36±0.2° for the treatment or control of macular edema.

In a further aspect, a pharmaceutical composition of the present invention may be adapted for administration by appropriate routes, for example by the oral (including buccal or sublingual), topical (including ophthalmic, otic, buccal, sublingual or transdermal), parenteral (including subcutaneous, intramuscular, intravenous or intradermal), intraocular (including intravitreal injection or implantation), or periocular route. Such compositions may be prepared by any method known in the art of pharmacy, for example by bringing into association a claimed molecular crystal of compound having Formula I (or a salt or an ester thereof) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

For treatments of the eye or other external tissues, for example skin, the compositions may be applied as a topical solution, suspension, emulsion, dispersion, ointment, or cream, as appropriate. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous vehicle.

Pharmaceutical compositions adapted for topical administration in the mouth include lozenges, pastilles and mouth washes.

Pharmaceutical compositions adapted for rectal administration may be presented as suppositories or as enemas.

Pharmaceutical compositions adapted for nasal administration wherein the carrier is a solid include a coarse powder having a particle size for example in the range 1 to 500 microns which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Preferred unit dosage compositions are those containing a daily dose or sub-dose, as herein above recited, or an appropriate fraction thereof, of an active ingredient.

In one embodiment, such a pharmaceutical composition comprises an aqueous carrier.

In another embodiment, such a pharmaceutical composition comprises an organic carrier, such as a hydrophobic or a hydrophilic organic material.

A suitable concentration is in the range from about 0.001 to about 10 percent (or alternatively, from about 0.01 to about 5 percent, or from about 0.01 to about 2 percent, or from about 0.01 to about 1 percent, or from about 0.001 to about 1 percent, or from about 0.05 to about 1 percent, or from about 0.05 to about 2 percent, or from about 0.1 to about 0.5 percent, from about 0.5 to about 1 percent, from about 1 to about 2 percent) by weight of the total composition is believed adequately to provide therapeutic value for treating or controlling pathological angiogenesis.

In one embodiment, a composition of the present invention is in a form of a suspension or dispersion. In another embodiment, the suspension or dispersion is based on an aqueous solution. For example, a composition of the present invention can comprise micrometer- or nanometer-sized particles of the active ingredient suspended or dispersed in sterile saline solution. In another embodiment, the suspension or dispersion is based on a hydrophobic medium. For example, the micrometer- or nanometer-sized (such as in the range from about 0.1 to about 10 μm) particles of the active ingredient (or a salt or ester thereof) can be suspended in a hydrophobic solvent e.g., silicone oil, mineral oil, or any other suitable nonaqueous medium for delivery to the eye. In still another embodiment, the micrometer- or nanometer-sized particles of the active ingredient (or a salt or ester thereof) can be coated with a physiologically acceptable surfactant (non-limiting examples are disclosed below), then the coated particles are dispersed in a liquid medium. The coating can keep the particles in a suspension. Such a liquid medium can be selected to produce a sustained-release suspension. For example, the liquid medium can be one that is sparingly soluble in the ocular environment into which the suspension is administered. In still another embodiment, the active ingredient (or a salt or ester thereof) is suspended or dispersed in a hydrophobic medium, such as an oil. In still another embodiment, such a medium comprises an emulsion of a hydrophobic material and water. In yet another embodiment, the insoluble active ingredient (or a salt or ester thereof) disclosed herein can be dosed by any normal drug delivery vehicle including but not limited to suspension in a liposome composition (both within and outside the liposome wall or strictly outside the liposome core), in the continuous phase of an emulsion or microemulsion, in the oil phase of the emulsion, or in a micellar solution using either charged or uncharged surfactants. A micellar solution wherein the surfactant is both the micelle forming agent and the anion of the active ingredient (or a salt or ester thereof) disclosed herein would be preferable.

In another aspect, a composition of the present invention can further comprise a non-ionic surfactant, such as polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp. 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006). The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1, or from about 0.01 to about 0.5 weight percent). Any of these surfactants also can be used to coat micrometer- or nanometer-sized particles, as disclosed above.

In addition, a composition of the present invention can include additives such as buffers, diluents, carriers, adjuvants, or other excipients. Any pharmacologically acceptable buffer suitable for application to the eye may be used. Other agents may be employed in the composition for a variety of purposes. For example, buffering agents, preservatives, co-solvents, oils, humectants, emollients, stabilizers, or antioxidants may be employed.

Water-soluble preservatives which may be employed include sodium bisulfate, sodium bisulfate, sodium thiosulfate, benzalkonium chloride, chlorobutanol, thimerosal, ethyl alcohol, methylparaben, polyvinyl alcohol, benzyl alcohol, phenylethyl alcohol, peroxide (such as hydrogen peroxide, urea hydrogen peroxide, or a source that generate a peroxide compound such as perborate), biguanide compounds, and quaternium compounds (such as polyquat-1, polyquat-10, etc.). These agents may be present in individual amounts of from about 0.001 to about 5 percent by weight (preferably, about 0.01 to about 2 percent by weight).

Suitable water-soluble buffering agents that may be employed are sodium carbonate, sodium borate, sodium phosphate, sodium acetate, sodium bicarbonate, etc., as approved by the United States Food and Drug Administration (“US FDA”) for the desired route of administration. These agents may be present in amounts sufficient to maintain a pH of the system of between about 5 and about 8. As such, the buffering agent may be as much as about 5 percent on a weight to weight basis of the total composition. Electrolytes such as, but not limited to, sodium chloride and potassium chloride may also be included in the composition. Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution.

Other buffers also may be found suitable or desirable in some circumstances, such as buffers based on HEPES (N-(2-hydroxyethyl)piperazine-N′-(2-ethanesulfonic acid)) having pKₐ of 7.5 at 25° C. and pH in the range of about 6.8-8.2; BES (N,N-bis(2-hydroxyethyl)2-aminoethanesulfonic acid) having pKₐ of 7.1 at 25° C. and pH in the range of about 6.4-7.8; MOPS (3-(N-morpholino)propanesulfonic acid) having pKₐ of 7.2 at 25° C. and pH in the range of about 6.5-7.9; TES (N-tris(hydroxymethyl)-methyl-2-aminoethanesulfonic acid) having pKₐ of 7.4 at 25° C. and pH in the range of about 6.8-8.2; MOBS (4-(N-morpholino)butanesulfonic acid) having pKₐ of 7.6 at 25° C. and pH in the range of about 6.9-8.3; DIPSO (3-(N,N-bis(2-hydroxyethyl)amino)-2-hydroxypropane)) having pKₐ of 7.52 at 25° C. and pH in the range of about 7-8.2; TAPSO (2-hydroxy-3(tris(hydroxymethyl)methylamino)-1-propanesulfonic acid)) having pKₐ of 7.61 at 25° C. and pH in the range of about 7-8.2; TAPS (((2-hydroxy-1,1-bis(hydroxymethyl)ethyl)amino)-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In one aspect, the composition has a pH that is suitable for administration into a subject; e.g., to render the composition non-irritating. For example, for topical ophthalmic administration, a desired pH is in the range from about 5 to about 8 (or alternatively from about 6 to about 7, or from about 6.4 to about 6.8).

In one aspect, the composition has a pH of about 7. Alternatively, the composition has a pH in a range from about 7 to about 7.5.

In another aspect, the composition has a pH of about 7.4.

In yet another aspect, a composition also can comprise a viscosity-modifying compound designed to facilitate the administration of the composition into the subject or to promote the bioavailability in the subject. In still another aspect, the viscosity-modifying compound may be chosen so that the composition is not readily dispersed after being administered into an ocular environment (such as the ocular surface, conjunctiva, or vitreous). Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 2000 centipoises (“cp” or mPa·s), measured with a Brookfield Laboratories cone-plate viscometer (model RVDV-III Ultra, spindle CPE-40) at 25° C., and shear rate of 7±1 sec⁻¹.

In another aspect, the present invention provides a method for producing a composition comprising the claimed stable molecular crystal of compound having Formula I (or a salt or ester thereof), the method comprising: (a) providing said stable molecular crystal of compound having Formula I (or a salt or ester thereof); and (b) dispersing an amount of said stable molecular crystal of compound having Formula I (or a salt or ester thereof) in a sufficient amount of said medium to produce said composition to achieve a predetermined concentration of said stable molecular crystal of compound having Formula I (or a salt or ester thereof) in said medium. Alternatively, a portion of stable molecular crystal of compound having Formula I (or a salt or ester thereof) remains in a solid phase for a period longer than 2 days, or 1 week, or 1 month, or 2 months, or 3 months, or 4 months, or 5 months, or 6 months, or 1 year, or 2 years after said stable molecular crystal of compound having Formula I (or a salt or ester thereof) has been in contact with said medium. In one embodiment, the method can optionally include a step of reducing the size of stable molecular crystal of compound having Formula I (or a salt or ester thereof) before dispersing such stable molecular crystal of compound having Formula I (or a salt or ester thereof) in the medium.

In still another aspect, the present invention provides a method for producing a stable molecular crystal of stable molecular crystal of compound having Formula I. The method comprises: (a) suspending a desired amount of a compound having Formula I in an aqueous solution to form a suspension; and (b) subjecting the suspension to an autoclaving condition to produce a stable molecular crystal of compound I, a XRPD spectrum of said stable molecular crystal exhibit peaks at 2θ angles of 7.44, 14.80, 16.64, and 23.04±0.2°.

In another aspect, said stable molecular crystal compound I, has a XRPD spectrum that exhibits peaks at 2θ angles of 7.44, 14.80, 16.64, 23.04, and 23.36±0.2°.

The method can further comprise recovering the stable molecular crystal of compound having Formula I with or without further drying said molecular crystal. The method can further comprise subjecting the recovered stable molecular crystal to a step of size reduction to nanometer- or micrometer-sized particles.

Some compositions of the present invention are disclosed in the examples below. It should be understood that the proportions of the listed ingredients may be adjusted for specific circumstances.

### Example 1

TABLE E-1

| Ingredient | Amount |
|---|---|
| Stable molecular crystal of | 60 mg |
| Compound having Formula I | |
| Polysorbate 80 | 3 mg |
| Phosphate buffer saline | q.s. to 1 g |

Predetermined amounts of ingredients according to the proportions in Table E-1 are charged into a stainless steel jacketed vessel that is equipped with a stirring mechanism. The mixture is vigorously stirred to produce the suspension. The final composition is sterilized, using, for example, heat or radiation and then packaged in appropriate containers.

### Example 2

A procedure similar to that disclosed in Example 1 is used to produce the composition of the present invention having the ingredients listed in Table E-2.

TABLE E-2

| Ingredient | Amount |
|---|---|
| Stable molecular crystal of | 30 mg |
| Compound having Formula I | |
| Miglyol 812 | q.s. to 1 g |

### Example 3

A procedure similar to that disclosed in Example 1 is used to produce the composition of the present invention having the ingredients listed in Table E-3.

TABLE E-3

| | Amount (% by weight, except where |
|---|---|
| Ingredient | “ppm” is indicated) |
| Glycerin | 3 |
| Propylene glycol | 3 |
| Stable molecular crystal of | 0.4 |
| Compound having Formula I | |
| Polyquat-1 | 1-10 ppm |
| Sunflower oil | q.s. to 100 |

### Example 4

A modification of the procedure disclosed in Example 1 is used to produce the composition of the present invention having the ingredients listed in Table E-4.

An appropriate proportion of polysorbate 80 (e.g., shown in Table E-4) is added to approximately 20 percent of the desired final volume of purified water in a stainless steel jacketed vessel that is equipped with a stirring mechanism. Glycerin and propylene glycol are then added to the mixture while mixing continues for five more minutes. To a sterilized second vessel, heated to about 80° C. and equipped with a stirring mechanism, containing approximately 70 percent of the desired final volume of purified water, an appropriate amount of CMC-MV is added over a period of three to five minutes while mixing continues until the CMC forms a substantially uniform solution. The contents of the second vessel are cooled to about room temperature and then the contents of the first vessel are transferred into the second vessel. The remaining of the desired volume of purified water is added to the second vessel. Then, an appropriate amounts of the claimed stable molecular crystal of Compound having Formula I is added to the contents of the second vessel over a period of three to five minutes while mixing continues until the drugs are substantially uniformly dispersed. The pH of the mixture is adjusted to 6.5-6.7 using 1 N NaOH. The final composition is sterilized, using, for example, heat or radiation, and packaged in appropriate containers.

TABLE 16

| | Amount (% by weight, except where |
|---|---|
| Ingredient | “ppm” is indicated) |
| Carboxymethyl cellulose, medium | 0.5 |
| viscosity (“CMC-MV”) | |
| Glycerin | 3 |
| Propylene glycol | 3 |
| Stable molecular crystal of | 0.6 |
| Compound having Formula I | |
| Polysorbate 80 ® (a surfactant) | 0.25 |
| Stabilized oxychloro complex | 20-50 ppm |
| Purified water | q.s. to 100 |

### Example 5

A procedure similar to that of Example 1 is used to produce a composition comprising the ingredients listed in Table E-5.

TABLE E-5

| | Amount (% by weight, except where |
|---|---|
| Ingredient | “ppm” is indicated) |
| Glycerin | 3 |
| Propylene glycol | 3 |
| Stable molecular crystal of | 0.5-6 |
| Compound having Formula I | |
| Tween ® 80 | 0.25 |
| Alexidine | 1-2 ppm |
| Corn oil | q.s. to 100 |

### Example 6

A procedure similar to that of Example 4 is used to produce a composition comprising the ingredients listed in Table E-6.

TABLE E-6

| | Amount (% by weight, except where |
|---|---|
| Ingredient | “ppm” is indicated) |
| CMC (MV) | 0.5 |
| Glycerin | 3 |
| Propylene glycol | 3 |
| Stable molecular crystal of | 0.5-6 |
| Compound having Formula I | |
| Tyloxapol (a surfactant) | 0.25 |
| Alexidine 2HCl | 1-2 ppm |
| Purified water | q.s. to 100 |

### Example 7

A procedure similar to that of Example 1 is used to produce a composition comprising the ingredients listed in Table E-7.

TABLE E-7

| | Amount (% by weight, except where |
|---|---|
| Ingredient | “ppm” is indicated) |
| HPMC | 0.5 |
| Glycerin | 3 |
| Propylene glycol | 3 |
| Stable molecular crystal of | 2 |
| Compound having Formula I | |
| Fluocinolone acetonide | 0.2 |
| Tyloxapol (a surfactant) | 0.25 |
| Benzakonium chloride | 100 ppm |
| Purified water | q.s. to 100 |

Alternatively, purified water may be substituted with an oil, such as fish oil, peanut oil, sesame oil, coconut oil, sunflower oil, corn oil, or olive oil to produce an oil-based composition comprising a stable molecular crystal of Compound having Formula I.

Fluocinolone acetonide may be replaced with another glucocorticosteroid (that is known as “safe steroid” having low risk of producing side effect, such as glaucoma) such as loteprednol (or a salt or ester thereof), or triamcinolone (or a salt or ester thereof).

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A molecular crystal form of a compound having Formula I, characterized by an X-ray powder diffraction (“XRPD”) spectrum that comprises peaks at 2θ angles of 7.44, 14.80, 16.64, and 23.04±0.2°

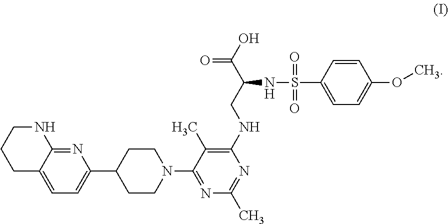

2. The molecular crystal form of compound having Formula I according to claim 1, wherein the XRPD spectrum further comprises a peak at 2θ angles of 23.36±0.2°.

3. The molecular crystal form of compound having Formula I according to claim 1, wherein said molecular crystal is formed by subjecting an aqueous suspension comprising a compound having Formula I to an autoclaving condition at 121-125° C., about 100-120 kPa above atmospheric pressure, for 30 minutes to 10 hours, under a closed atmosphere generated by said aqueous suspension.

4. The molecular crystal form of compound having Formula I according to claim 1, wherein said molecular crystal is substantially incapable of changing in crystalline structure, as exhibited by a plurality of peaks in an X-ray powder diffraction (“XRPD”) spectrum, upon storage at normal room conditions of temperature, pressure, and humidity after at least 1 month, as exhibited by a relative change of less than about 5 percent in the peak height of the highest peak in its XRPD spectrum, wherein said normal room conditions of temperature, pressure, and humidity are 20-28° C., 95-105 kPa, and 20-80% relative humidity.

5. A pharmaceutical composition comprising a molecular crystal form of a compound having Formula I, 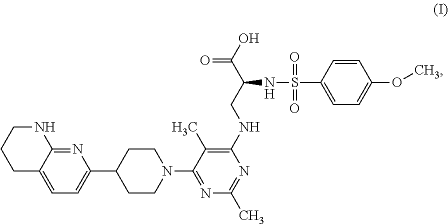
wherein said molecular crystal is characterized by an X-ray powder diffraction (“XRPD”) spectrum that comprises peaks at 2θ angles of 7.44, 14.80, 16.64, and 23.04±0.2°.

6. The pharmaceutical composition of claim 5, further comprising an ophthalmically acceptable carrier, wherein said composition is suitable for intravitreal administration.

7. A method for treating or controlling a disease resulting from a pathological angiogenesis, said method comprising administering to a subject, who suffers from, or is at risk of developing, said disease, a pharmaceutical composition comprising a molecular crystal form of a compound having Formula I, characterized by an X-ray powder diffraction (“XRPD”) spectrum that comprises peaks at 2θ angles of 7.44, 14.80, 16.64, and 23.04±0.2°

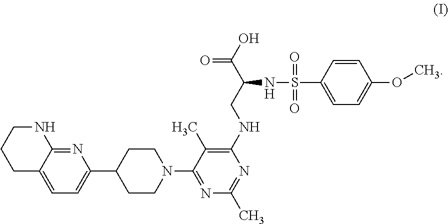

8. The method of claim 7, wherein said pharmaceutical composition further comprises an ophthalmically acceptable carrier.

9. The method of claim 8, wherein said disease is selected from the group consisting of wet age-related macular degeneration, dry age-related macular degeneration, macular edema, diabetic retinopathy, and combinations thereof.

10. The method of claim 9, wherein said disease comprises wet age-related macular degeneration.

11. The method of claim 10, wherein said pharmaceutical composition is administered intravitreally to said subject.

12. A method of producing a molecular crystal of a compound having Formula I, 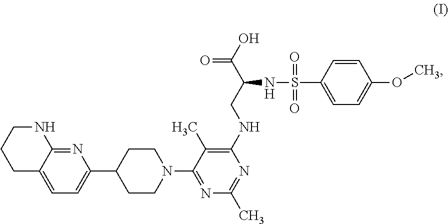
the method comprising subjecting a suspension comprising a compound having Formula I to an autoclaving condition at 121-125° C., about 100-120 kPa above atmospheric pressure, for 30 minutes to 10 hours, under a closed atmosphere generated by said aqueous suspension, wherein said molecular crystal is characterized by an X-ray powder diffraction (“XRPD”) spectrum that comprises peaks at 2θ angles of 7.44, 14.80, 16.64, and 23.04±0.2°.

13. The method of claim 12, wherein said molecular crystal is substantially incapable of changing in crystalline structure, as exhibited by a plurality of peaks in an X-ray powder diffraction (“XRPD”) spectrum, upon storage at normal room conditions of temperature, pressure, and humidity after at least 1 month, as exhibited by a relative change of less than about 5 percent in the peak height of the highest peak in its XRPD spectrum, wherein said normal room conditions of temperature, pressure, and humidity are 20-28° C., 95-105 kPa, and 20-80% relative humidity.
